(19) United States
(12) Patent Application Publication
(10) Pub. No.: US 2019/0312205 A1
(43) Pub. Date: Oct. 10, 2019

(54) A METHOD OF MANUFACTURING AN ORGANIC LIGHT-EMITTING DEVICE

(71) Applicant: Sumitomo Chemical Company Limited, Tokyo (JP)

(72) Inventors: Matthew Stevenson, Godmanchester (GB); Ian Johnson, Godmanchester (GB); Simon King, Godmanchester (GB)

(73) Assignee: Sumitomo Chemical Company Limited, Tokyo (JP)

(21) Appl. No.: 16/463,219

(22) PCT Filed: Nov. 16, 2017
(86) PCT No.: PCT/GB2017/053453

§ 371 Date: May 22, 2019

(30) Foreign Application Priority Data
Nov. 22, 2016 (GB) 1619740.2

(51) Int. Cl. H01L 51/00

(52) U.S. Cl. CPC H01L 51/0026; H01L 51/0074; H01L 51/5096; H01L 51/0073; H01L 51/0085

(57) ABSTRACT

A method of forming an organic light-emitting device comprising the steps of: forming a first light-emitting layer over an anode; forming a layer comprising a compound of formula (I) on the light-emitting layer to form a partially formed device: Formula (I) wherein Ar in each occurrence is independently a C₆₋₂₀ aromatic group; m is at least 1; n is at least 1; and X is a group of formula (II): wherein Y is O or S; R¹ in each occurrence is independently a substituent; p is 0 or a positive integer; and * is a bond to Ar; forming a cathode over the compound of formula (I); and heating the partially formed device before and/or after formation of the cathode.

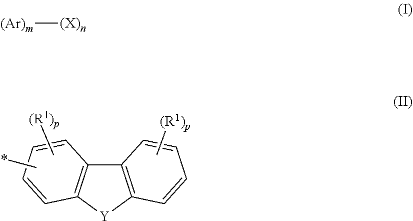

### FIELD OF THE INVENTION

The invention relates to a method of forming an organic light-emitting device.

### BACKGROUND OF THE INVENTION

Electronic devices containing active organic materials are known for use in devices such as organic light emitting diodes (OLEDs), organic photoresponsive devices (in particular organic photovoltaic devices and organic photosensors), organic transistors and memory array devices. Devices containing active organic materials offer benefits such as low weight, low power consumption and flexibility. Moreover, use of soluble organic materials allows use of solution processing in device manufacture, for example inkjet printing or spin-coating.

An organic light-emitting device has a substrate carrying an anode, a cathode and an organic light-emitting layer containing a light-emitting material between the anode and cathode.

In operation, holes are injected into the device through the anode and electrons are injected through the cathode. Holes in the highest occupied molecular orbital (HOMO) and electrons in the lowest unoccupied molecular orbital (LUMO) of the light-emitting material combine to form an exciton that releases its energy as light.

Layers other than the organic light-emitting layer may be provided between the anode and cathode to enhance device performance.

WO 2009/085344 discloses compounds for use as a host in a light-emitting layer containing the host and an emissive dopant or in an enhancement layer.

It is an object of the invention to provide long lifetime organic light-emitting devices.

### SUMMARY OF THE INVENTION

The present inventors have surprisingly found that the lifetime of an organic light-emitting device may be increased by providing a layer of certain dibenzothiophene or dibenzofuran materials on a light-emitting layer of the device followed by heating.

Accordingly, in a first aspect the invention provides a method of forming an organic light-emitting device comprising the steps of:

forming a first light-emitting layer over an anode;
forming a layer comprising a compound of formula (I) on the light-emitting layer to form a partially formed device:

(Ar)ₘ—(X)ₙ (I)

wherein Ar in each occurrence is independently a C₆₋₂₀ aromatic group; m is at least 1; n is at least 1; and X is a group of formula (II):

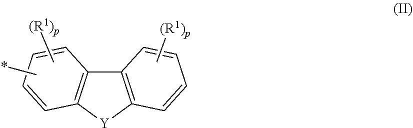

wherein Y is O or S; R¹ in each occurrence is independently a substituent; p is 0 or a positive integer; and * is a bond to Ar;
   forming a cathode over the compound of formula (I); and
   heating the partially formed device before and/or after formation of the cathode.

A layer formed “on” an underlying layer as described herein is adjacent to the underlying layer.

A layer formed “over” an underlying layer as described herein is adjacent to the underlying layer or spaced apart therefrom by one or more intervening layers.

### DESCRIPTION OF THE DRAWINGS

The invention will now be described in more detail with reference to the drawings in which:

FIG. 1 is a graph of luminance vs. time for an OLED formed according to a method according to an embodiment of the invention compared to a comparative OLED.

### DETAILED DESCRIPTION OF THE INVENTION

An OLED as described herein is formed by separately depositing a first light-emitting layer and a layer comprising a compound of formula (I) followed by heating of the light-emitting layer and the layer comprising the compound of formula (I).

Preferably, heating is carried out at above the glass transition temperature of at least one of a component of the first light-emitting layer and the compound of formula (I).

Formula (I) is:

(Ar)ₘ—(X)ₙ (I)

wherein Ar in each occurrence is independently a C₆₋₂₀ aromatic group which may be unsubstituted or substituted with one or more substituents; m is at least 1; n is at least 1; and X is a group of formula (II):

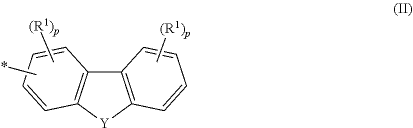

wherein Y is O or S; R¹ in each occurrence is independently a substituent; p is 0 or a positive integer; and * is a direct bond to Ar.

Preferably m is 1 or 2.

Preferably n is 1 or 2.

If present, substituents R¹, which may be the same or different in each occurrence, are optionally selected from F; CN; NO₂; and C₁₋₂₀ alkyl wherein one or more non-adjacent, non-terminal C atoms of the alkyl group may be replaced with O, S, NR² or SiR²₂, COO or CO wherein R² in each occurrence is a C₁₋₂₀ hydrocarbyl group, optionally a C₁₋₁₂ alkyl group, unsubstituted phenyl, or phenyl substituted with one or more alkyl groups.

A “non-terminal carbon atom” of an alkyl group as used herein means carbon atoms other than the methyl group of a n-alkyl chain or the methyl groups of a branched alkyl chain.

Preferably, compounds of formula (I) are selected from formulae (Ia)-(Id):

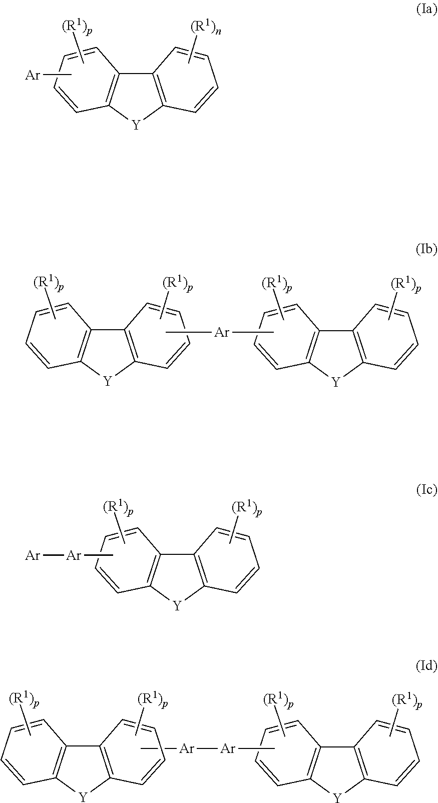

Preferably, the group of formula (II) has formula (IIa):

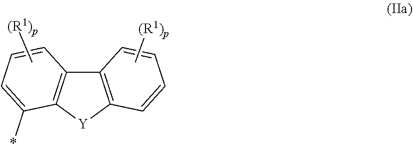

Preferably, Ar is selected from fluorene and phenyl, each of which may be unsubstituted or substituted with one or more substituents, more preferably from groups of formula (IIIa)-(IIIf):

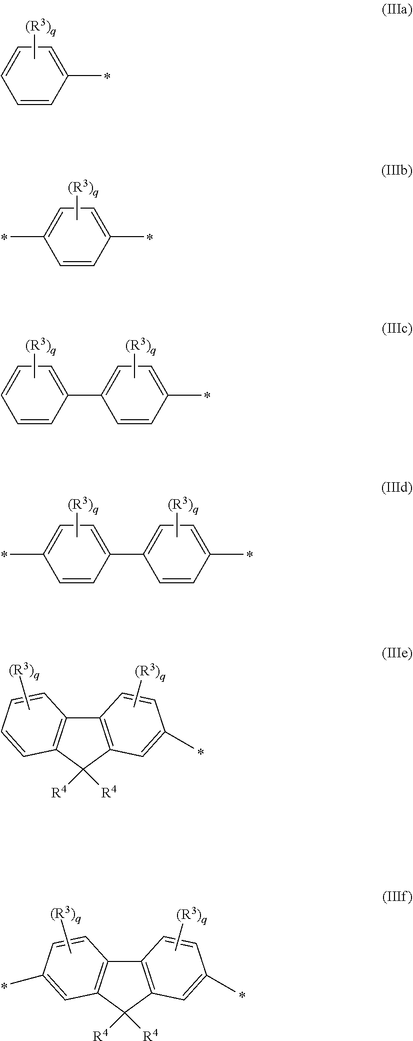

wherein R³ independently in each occurrence is a substituent; q is 0 or a positive integer, optionally 1, 2 or 3; and R⁴ independently in each occurrence is a substituent wherein the two groups R⁴ may be linked to form an unsubstituted or substituted ring.

Optionally, each R³ (if present) may independently be selected from the group consisting of F; CN; NO₂; and C₁₋₂₀ alkyl wherein one or more non-adjacent, non-terminal C atoms of the alkyl group may be replaced with O, S, NR² or SiR²₂, COO or CO wherein R² in each occurrence is a C₁₋₂₀ hydrocarbyl group, optionally a C₁₋₁₂ alkyl group, unsubstituted phenyl, or phenyl substituted with one or more alkyl groups.

Optionally, each R⁴ is independently be selected from the group consisting of C₁₋₂₀ alkyl wherein one or more non-adjacent, non-terminal C atoms of the alkyl group may be replaced with O, S, NR² or SiR²₂, COO or CO; and a group of formula (Ar¹)ᵣ wherein r is at least 1, optionally 1, 2 or 3, and Ar¹ is an aryl or heteroaryl group, preferably phenyl, which may be unsubstituted or substituted with one or more substituents; and wherein R² in each occurrence is a C₁₋₂₀ hydrocarbyl group, optionally a C₁₋₁₂ alkyl group, unsubstituted phenyl, or phenyl substituted with one or more alkyl groups.

Substituents of Ar¹, if present, may be selected from F; CN; NO₂; and C₁₋₂₀ alkyl wherein one or more non-adjacent, non-terminal C atoms of the alkyl group may be replaced with O, S, NR² or SiR²₂, COO or CO wherein R² in each occurrence is a C₁₋₂₀ hydrocarbyl group, optionally a C₁₋₁₂ alkyl group, unsubstituted phenyl, or phenyl substituted with one or more alkyl groups.

Preferably, q is 0.

Exemplary compounds of formula (I) include the following:

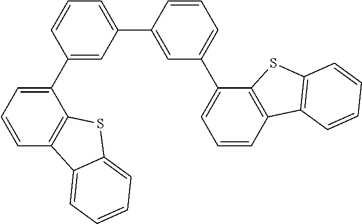 [SMILES: c1cc(-c2cccc(-c3cccc4c3sc3ccccc34)c2)cc(-c2cccc3c2sc2ccccc23)c1]

Preferably, compounds of formula (I) have a glass transition temperature in the range of 70-150° C., optionally 70-100° C.

Glass transition temperatures as described herein are as measured using a PerkinElmer DSC8500 differential scanning calorimeter with autosampler.

The first light-emitting layer may be deposited directly onto an anode. It is preferred that at least one layer is provided between the anode and the first light-emitting layer, more preferably one or both of a hole-transporting layer and a hole injection layer.

In a particularly preferred embodiment, a hole-injection layer is provided between the anode and the first light-emitting layer and a hole-transporting layer is provided between the hole-injection layer and the first light-emitting layer.

The first light-emitting layer preferably comprises or consists of a host material and a light-emitting dopant, preferably a phosphorescent light-emitting dopant.

A layer comprising or consisting of a compound of formula (I) is deposited on the first light-emitting layer to form a partially-formed device. The layer may be deposited by any method disclosed herein, including solution deposition and evaporation methods, preferably by evaporation.

A cathode is formed over the layer comprising the compound of formula (I). The cathode may be formed directly on the layer comprising the compound of formula (I) or at least one intervening layer may be formed.

Preferably, an electron transporting layer or an electron injecting layer is formed between the compound of formula (I) and the cathode. Preferably, the electron-transporting layer or electron injecting layer is the only layer between the first light-emitting layer and the cathode.

The device stack comprising or consisting of the partially formed device is heated before and/or after formation of the cathode.

Preferably, an electron-transporting layer or electron injection layer is deposited over the partially formed device and heated before formation of the cathode.

Preferably, heating is at or above the glass transition temperature of at least one of a component of the first light-emitting layer and the compound of formula (I), more preferably at a temperature above the glass transition temperature of both a component of the first light-emitting layer and the compound of formula (I).

The device stack may be heated by any suitable apparatus, for example a hotplate. The device stack may be heated to a temperature in the range of 80-180° C., optionally in the range of 100-160° C. or 100-150° C.

The OLED may be a display, optionally a full-colour display wherein the first light-emitting layer comprises pixels comprising red, green and blue subpixels.

The OLED may be a white-emitting OLED. White-emitting OLEDs as described herein may have a CIE x coordinate equivalent to that emitted by a black body at a temperature in the range of 2500-9000K and a CIE y coordinate within 0.05 or 0.025 of the CIE y coordinate of said light emitted by a black body, optionally a CIE x coordinate equivalent to that emitted by a black body at a temperature in the range of 2700-6000K. A white-emitting OLED may contain a plurality of light-emitting materials, preferably red, green and blue light-emitting materials, more preferably red, green and blue phosphorescent light-emitting materials, that combine to produce white light. The light-emitting materials may all be provided in the first light-emitting layer described hereinbefore, or one or more further light-emitting layers which emit light when the device is in use may be present.

A red light-emitting material may have a photoluminescence spectrum with a peak in the range of about more than 550 up to about 700 nm, optionally in the range of about more than 560 nm or more than 580 nm up to about 630 nm or 650 nm.

A green light-emitting material may have a photoluminescence spectrum with a peak in the range of about more than 490 nm up to about 560 nm, optionally from about 500 nm, 510 nm or 520 nm up to about 560 nm.

A blue light-emitting material may have a photoluminescence spectrum with a peak in the range of up to about 490 nm, optionally about 450-490 nm.

Photoluminescence spectra described herein are as measured by casting 5 wt % of the material in a polystyrene film onto a quartz substrate and measuring in a nitrogen environment using apparatus C9920-02 supplied by Hamamatsu.

### Anode

At least one of the anode and the cathode is transparent. Preferably the anode is transparent. Suitable transparent anode materials are transparent metal oxides, preferably indium tin oxide or indium zinc oxide. In other embodiments, the anode may be an opaque material and the cathode may be transparent.

The device is supported on a substrate. In the case of a transparent anode, the substrate is selected from a transparent material, optionally glass or one or more layers of a transparent material, optionally a transparent plastic.

If the anode is opaque then the substrate may be opaque or transparent, and light may be emitted through a transparent cathode.

### First Light-Emitting Layer

The first light-emitting layer contains at least one light-emitting material. The first light-emitting layer may consist of a single light-emitting material or may be a mixture of more than one material, optionally a host doped with one or more light-emitting dopants.

The or each light-emitting material of the first light-emitting layer may be a fluorescent material or a phosphorescent material. Light-emitting materials may be selected from polymeric and non-polymeric light-emitting materials. Exemplary light-emitting polymers are conjugated polymers, for example polyphenylenes and polyfluorenes examples of which are described in Bernius, M. T., Inbasekaran, M., O'Brien, J. and Wu, W., Progress with Light-Emitting Polymers. Adv. Mater., 12 1737-1750, 2000, the contents of which are incorporated herein by reference.

A fluorescent light-emitting layer may consist of a light-emitting material alone or may further comprise one or more further materials mixed with the light-emitting material. Exemplary further materials may be selected from hole-transporting materials; electron-transporting materials and triplet-accepting materials, for example a triplet-accepting polymer as described in WO 2013/114118, the contents of which are incorporated herein by reference.

Preferably, the first light-emitting layer comprises a host material and at least one light-emitting dopant, preferably a phosphorescent dopant, that emits light when the device is in operation. Preferably, all light emitted by the device when in operation is phosphorescence.

Preferably, the host has a LUMO of at least 1.8 eV or at least 1.9 eV from vacuum level.

Preferably, the host has a LUMO of no more than 2.1 eV or no more than 2.0 eV from vacuum level.

Preferably, the difference in LUMO level of the host material and the compound of formula (I) is no more than about 0.2 eV, optionally no more than about 0.1 eV.

Preferably, the host has a HOMO level of 5.5-6.2 eV or 5.9-6.2 eV from vacuum level.

Preferably, the difference in HOMO level of the host material and the compound of formula (I) is no more than about 0.2 eV, optionally no more than about 0.1 eV.

Exemplary phosphorescent dopants are row 2 or row 3 transition metal complexes.

Exemplary phosphorescent light-emitting compounds have formula (IV):

ML¹ₓL²_{y}L³_{z} (IV)

wherein M is a metal; each of L¹, L² and L³ is a coordinating group that independently may be unsubstituted or substituted with one or more substituents; x is a positive integer; y and z are each independently 0 or a positive integer; and the sum of (a. x)+(b. y)+(c.z) is equal to the number of coordination sites available on M, wherein a is the number of coordination sites on L¹, b is the number of coordination sites on L² and c is the number of coordination sites on L³.

a, b and c are preferably each independently 1, 2 or 3. Preferably, a, b and c are each a bidentate ligand (a, b and c are each 2). In a preferred embodiment, x is 3 and y and z are 0. In another preferred embodiment, x is 1 or 2, y is 1 and z is 0 or 1.

M is preferably selected from ruthenium, rhodium, palladium, rhenium, osmium, iridium, platinum and gold, preferably Ir³⁺.

Exemplary ligands L¹, L² and L³ include carbon or nitrogen donors such as porphyrin or bidentate ligands of formula (V):

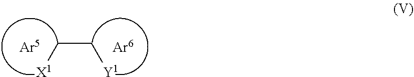

wherein Ar⁵ and Ar⁶ may be the same or different and are independently selected from substituted or unsubstituted aryl or heteroaryl; X¹ and Y¹ may be the same or different and are independently selected from carbon or nitrogen; and Ar⁵ and Ar⁶ may be fused together. Ligands wherein X¹ is carbon and Y¹ is nitrogen are preferred, in particular ligands in which Ar⁵ is a monocyclic or fused heteroaromatic group, optionally a 5-20 membered heteroaromatic group, of N and C atoms only for example pyridyl or isoquinoline, and Ar⁶ is a monocyclic or fused aromatic group, optionally a C6-20 aromatic group for example phenyl or naphthyl.

To achieve red emission, Ar⁵ may be selected from phenyl, fluorene, naphthyl and Ar⁶ are selected from quinoline, isoquinoline, thiophene and benzothiophene.

To achieve green emission, Ar⁵ may be selected from phenyl or fluorene and Ar⁶ may be pyridine.

To achieve blue emission, Ar⁵ may be selected from phenyl and Ar⁶ may be selected from imidazole, pyrazole, triazole and tetrazole.

Examples of bidentate ligands are illustrated below:

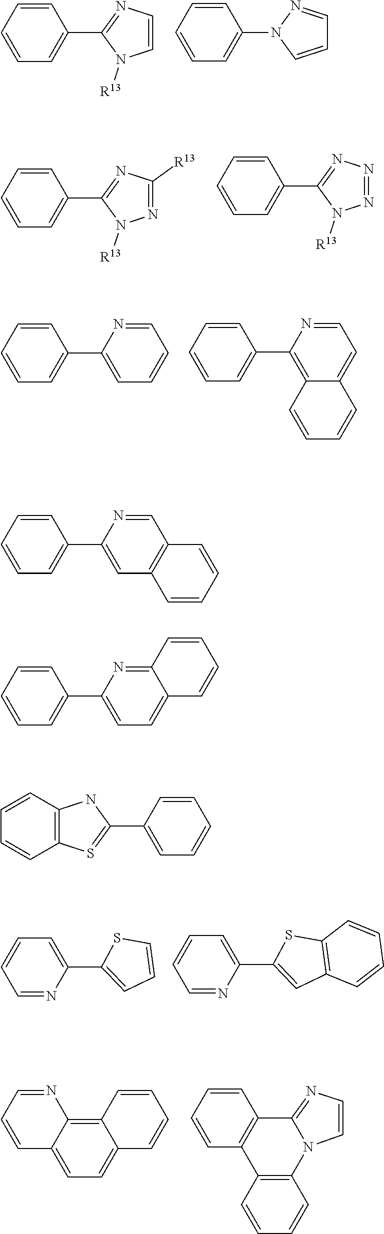

wherein R¹³ is a substituent, preferably a C₁₋₂₀ hydrocarbyl group, optionally C₁₋₁₂ alkyl, unsubstituted phenyl or phenyl substituted with one or more C₁₋₁₂ alkyl groups.

Each of Ar⁵ and Ar⁶ may independently be unsubstituted or may be substituted with one or more substituents. Two or more of these substituents may be linked to form a ring, for example an aromatic ring.

Substituents or Ar⁵ and/or Ar⁶ may be selected from:

### F;

### CN;

### NO₂;

C₁₋₂₀ alkyl wherein one or more non-adjacent, non-terminal C atoms of the alkyl group may be replaced with O, S, NR² or SiR²₂, COO or CO wherein R² in each occurrence is a C₁₋₂₀ hydrocarbyl group, optionally a C₁₋₁₂ alkyl group, unsubstituted phenyl, or phenyl substituted with one or more alkyl groups;
an aryl or diaryl group which may be unsubstituted or substituted with one or more substituents, optionally phenyl or biphenyl which may be unsubstituted or substituted with one or more C₁₋₁₂ alkyl groups or alkoxy groups; and
dendrons.

A dendron comprises a branching point bound to the ligand of formula (V) and two or more dendritic branches. Preferably, the dendron is at least partially conjugated, and at least one of the branching points and dendritic branches comprises an aryl or heteroaryl group, for example a phenyl group. In one arrangement, the branching point group and the branching groups are all phenyl, and each phenyl may independently be substituted with one or more substituents, for example alkyl or alkoxy.

A dendron may have optionally substituted formula (XI)

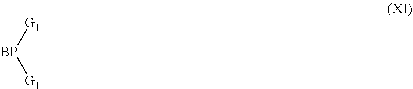

wherein BP represents a branching point for attachment to a core and G₁ represents first generation branching groups.

The dendron may be a first, second, third or higher generation dendron. G₁ may be substituted with two or more second generation branching groups G₂, and so on, as in optionally substituted formula (XIa):

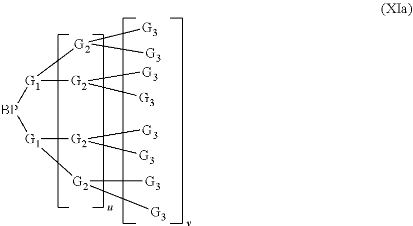

wherein u is 0 or 1; v is 0 if u is 0 or may be 0 or 1 if u is 1; BP represents a branching point for attachment to a core and G₁, G₂ and G₃ represent first, second and third generation dendron branching groups. In one preferred embodiment, each of BP and G₁, G₂ . . . Gₙ is phenyl, and each phenyl BP, G₁, G₂ . . . Gₙ₋₁ is a 3,5-linked phenyl.

A preferred dendron is a substituted or unsubstituted dendron of formula (XIb) and (XIc):

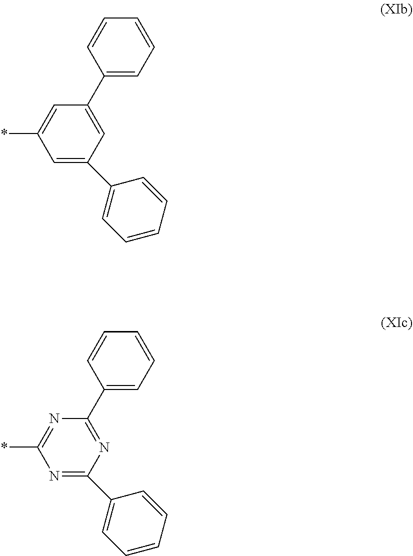 [SMILES: *c1cc(-c2ccccc2)cc(-c2ccccc2)c1 ; *c1nc(-c2ccccc2)nc(-c2ccccc2)n1]

wherein * represents an attachment point of the dendron to a core.

BP and/or any group G may be substituted with one or more substituents, for example one or more C₁₋₂₀ alkyl or alkoxy groups.

Other ligands suitable for use with d-block elements include N,N-bidentate ligands, optionally bipyridyl; N,O-bidentate ligands, optionally picolinate; and O,O-bidentate ligands, optionally diketonates, in particular acetylacetonate (acac), tetrakis-(pyrazol-1-yl)borate, 2-carboxypyridyl, triarylphosphines and pyridine, each of which may be substituted.

One or more of L¹, L² and L³ may comprise a carbene group.

Light-emitting dopant(s) of a first light-emitting layer comprising a host material and one or more light-emitting dopants may make up about 0.05 wt % up to about 45 wt %, optionally about 1-40 wt % of the light-emitting layer.

Preferably, the first light-emitting layer comprises at least one light-emitting material having a HOMO of at least 0.3 eV, optionally at least 0.5 eV, closer to vacuum level than that of either the host material or the compound of formula (I).

Preferred host materials are hosts comprising a dibenzothiophene or dibenzofuran group.

The host material may have formula (VI):

(Ar³)_{f}—(Z)_{g} (VI)

wherein:
Ar³ is a C₆₋₂₀ aromatic group which may be unsubstituted or substituted with one or more substituents; f is at least 1; g is at least 1; and Z in each occurrence is independently a group of formula (VII):

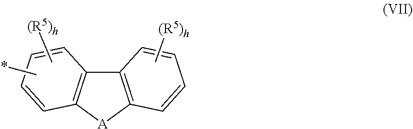

wherein A is S or O;
R⁵ independently in each occurrence is a substituent; and each h is independently 0 or a positive integer.

Preferably, f is 1 or 2

Preferably, g is 1 or 2.

Preferably, h is 0, 1, 2 or 3.

If present, substituents R⁵, which may be the same or different in each occurrence, are optionally selected from F; CN; NO₂; C₆₋₂₀ aryl, preferably phenyl, which may be unsubstituted or substituted with one or more substituents; and C₁₋₂₀ alkyl wherein one or more non-adjacent, non-terminal C atoms of the alkyl group may be replaced with O, S, NR² or SiR²₂, COO or CO wherein R² in each occurrence is a C₁₋₂₀ hydrocarbyl group.

R², if present, is preferably a C₁₋₁₂ alkyl group, unsubstituted phenyl, or phenyl substituted with one or more alkyl groups.

Substituents of a C₆₋₂₀ aryl group R⁵, if present, are optionally selected from C₁₋₁₂ alkyl and C₁₋₁₂ alkoxy.

Preferably, Ar³ is selected from the group consisting of fluorene and phenyl. More preferably, f is 1 and Ar3 is fluorene, or f is 1 or 2 and each Ar3 is phenyl.

Preferably, —(Ar³)_{f} may be selected from formulae (VIIIa)-(VIIIe):

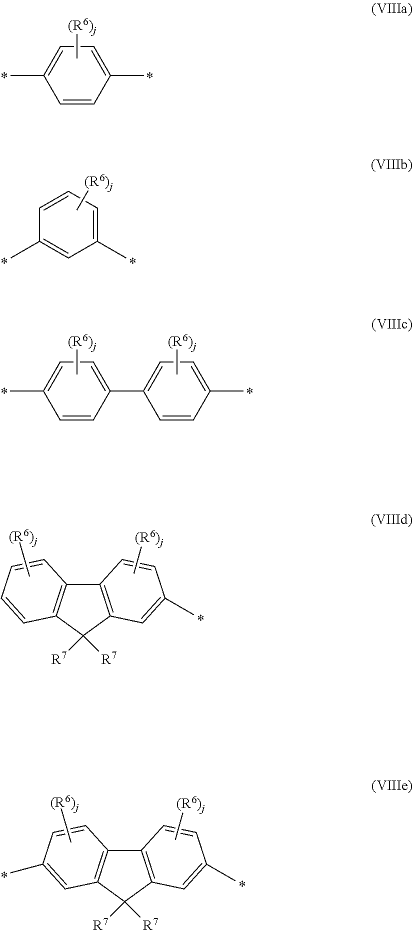

wherein R⁶ independently in each occurrence is a substituent; j is 0 or a positive integer, optionally 1, 2 or 3; and R⁶ independently in each occurrence is a substituent wherein the two groups R⁶ may be linked to form an unsubstituted or substituted ring.

Optionally, each R⁶ (if present) may independently be selected from the group consisting of F; CN; NO₂; and C₁₋₂₀ alkyl wherein one or more non-adjacent, non-terminal C atoms of the alkyl group may be replaced with O, S, NR² or SiR²₂, COO or CO wherein R² in each occurrence is a C₁₋₂₀ hydrocarbyl group, optionally a C₁₋₁₂ alkyl group, unsubstituted phenyl, or phenyl substituted with one or more alkyl groups.

Optionally, each R⁷ is independently be selected from the group consisting of C₁₋₂₀ alkyl wherein one or more non-adjacent, non-terminal C atoms of the alkyl group may be replaced with O, S, NR² or SiR²₂, COO or CO; and a group of formula (Ar⁴)ₜ wherein t is at least 1, optionally 1, 2 or 3, and Ar⁴ is an aryl or heteroaryl group, preferably phenyl, which may be unsubstituted or substituted with one or more substituents; and wherein R² in each occurrence is a C₁₋₂₀ hydrocarbyl group, optionally a C₁₋₁₂ alkyl group, unsubstituted phenyl, or phenyl substituted with one or more alkyl groups.

Substituents of Ar⁴, if present, may be selected from F; CN; NO₂; and C₁₋₂₀ alkyl wherein one or more non-adjacent, non-terminal C atoms of the alkyl group may be replaced with O, S, NR² or SiR²₂, COO or CO wherein R² in each occurrence is a C₁₋₂₀ hydrocarbyl group, optionally a C₁₋₁₂ alkyl group, unsubstituted phenyl, or phenyl substituted with one or more alkyl groups.

Preferably, j is 0.

The host may have formula (IX) wherein A is O or S:

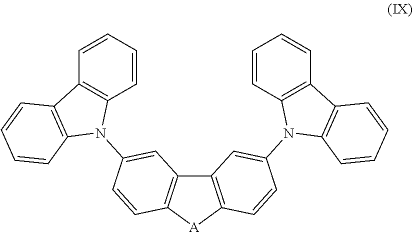

The host of formula (IX) may be unsubstituted or may be substituted with one or more substituents R⁶ wherein R⁶ is as described above.

The first light-emitting layer preferably contains at least one material having a glass transition temperature in the range of 70-150° C., optionally 90-140° C. or 110-140° C. This material is preferably a host material. This material is preferably a major component of the first light-emitting layer. By “major component” is meant a component making up at least 50 weight % of the mass of the first light-emitting layer.

### Electron Transporting and Electron Injecting Layers

An electron-transporting material for forming an electron-transporting layer as described herein comprising or consisting of the electron-transporting material preferably has a LUMO in the range of about 1.8-2.5 eV. Suitable electron-transporting materials include, without limitation, a polymer comprising one or more arylene repeat units. Exemplary arylene repeat units may be selected from fluorene, phenylene and anthracene repeat units. Exemplary electron-transporting polymers are disclosed in WO 2012/133229, the contents of which are incorporated herein.

An electron-injecting layer may comprise an n-doped electron-transporting material.

The n-dopant may be a 2,3-dihydro-1H-benzoimidazole, optionally 1,3-Dimethyl-2-phenyl-2,3-dihydro-1H-benzoimidazole (DMBI) or 4-(2,3-Dihydro-1,3-dimethyl-1H-benzimidazol-2-yl)-N,N-dimethylbenzenamine (N-DMBI).

### Cathode

The cathode comprises at least one layer comprising a conductive material. Exemplary conductive materials are metals, preferably metals having a work function of at least 4 eV, optionally aluminium, copper, silver or gold or iron. Exemplary non-metallic conductive materials include conductive metal oxides, for example indium tin oxide and indium zinc oxide, graphite and graphene. Work functions of metals are as given in the CRC Handbook of Chemistry and Physics, 12-114, 87^{th} Edition, published by CRC Press, edited by David R. Lide. If more than one value is given for a metal then the first listed value applies.

The cathode may consist of a single conductive layer. In one embodiment, the OLED comprises a cathode consisting of a single conductive layer in direct contact with an electron injecting layer.

The cathode may comprise two or more layers. In a preferred embodiment the cathode comprises a first conductive layer and a metal compound layer between the first conductive layer and the organic layers of the OLED. Preferably, a first surface of the metal compound layer is in contact with an organic layer of the OLED, preferably an electron-transporting layer, and a second, opposing surface of the metal compound layer is in contact with the first conductive layer.

Preferably, the metal compound is an alkali or alkali earth compound. Preferably, the metal compound is a halide, more preferably a fluoride. Exemplary metal compounds include, without limitation, lithium fluoride, sodium fluoride, potassium fluoride, rubidium fluoride, cesium fluoride, beryllium fluoride, magnesium fluoride, calcium fluoride, strontium fluoride and barium fluoride. Alkali metal fluorides are particularly preferred.

The cathode may be opaque or transparent. Transparent cathodes are particularly advantageous for active matrix devices because emission through a transparent anode in such devices is at least partially blocked by drive circuitry located underneath the emissive pixels.

### Hole-Transporting Layer

A hole transporting layer may be provided between the anode and the light-emitting layer.

The hole-transporting layer may be cross-linked, particularly if an overlying layer is deposited from a solution. The crosslinkable group used for this crosslinking may be a crosslinkable group comprising a reactive double bond such and a vinyl or acrylate group, or a benzocyclobutane group. Crosslinking may be performed by thermal treatment, preferably at a temperature of less than about 250° C., optionally in the range of about 100-250° C.

A hole transporting layer may comprise or may consist of a hole-transporting polymer, which may be a homopolymer or copolymer comprising two or more different repeat units. The hole-transporting polymer may be conjugated or non-conjugated. Exemplary conjugated hole-transporting polymers are polymers comprising arylamine repeat units, for example as described in WO 99/54385 or WO 2005/049546 the contents of which are incorporated herein by reference. Conjugated hole-transporting copolymers comprising arylamine repeat units may have one or more co-repeat units selected from arylene repeat units, for example one or more repeat units selected from fluorene, phenylene, phenanthrene naphthalene and anthracene repeat units, each of which may independently be unsubstituted or substituted with one or more substituents, optionally one or more C₁₋₄₀ hydrocarbyl substituents.

If present, a hole transporting layer located between the anode and the light-emitting layer 105 preferably has a HOMO level of less than or equal to 5.5 eV, more preferably around 4.8-5.5 eV or 5.1-5.3 eV as measured by square wave voltammetry. The HOMO level of the hole transport layer may be selected so as to be within 0.2 eV, optionally within 0.1 eV, of a material in an adjacent layer in order to provide a small barrier to hole transport between these layers.

Preferably a hole-transporting layer, more preferably a crosslinked hole-transporting layer, is adjacent to the first light-emitting layer.

A hole-transporting layer may consist essentially of a hole-transporting material or may comprise one or more further materials. A light-emitting material, optionally a phosphorescent material, may be provided in the hole-transporting layer.

A phosphorescent material may be covalently bound to a hole-transporting polymer as a repeat unit in the polymer backbone, as an end-group of the polymer, or as a side-chain of the polymer. If the phosphorescent material is provided in a side-chain then it may be directly bound to a repeat unit in the backbone of the polymer or it may be spaced apart from the polymer backbone by a spacer group. Exemplary spacer groups include C₁₋₂₀ alkyl and aryl-C₁₋₂₀ alkyl, for example phenyl-C₁₋₂₀ alkyl. One or more carbon atoms of an alkyl group of a spacer group may be replaced with O, S, C═O or COO.

Emission from a light-emitting hole-transporting layer and emission from the first light-emitting layer may combine to produce white light.

### Hole Injection Layers

A conductive hole injection layer, which may be formed from a conductive organic or inorganic material, may be provided between the anode and the first light-emitting layer. Examples of doped organic hole injection materials include optionally substituted, doped poly(ethylene dioxythiophene) (PEDT), in particular PEDT doped with a charge-balancing polyacid such as polystyrene sulfonate (PSS) as disclosed in EP 0901176 and EP 0947123, polyacrylic acid or a fluorinated sulfonic acid, for example Nafion®; polyaniline as disclosed in U.S. Pat. Nos. 5,723,873 and 5,798,170; and optionally substituted polythiophene or poly(thienothiophene). Examples of conductive inorganic materials include transition metal oxides such as VOx MoOx and RuOx as disclosed in Journal of Physics D: Applied Physics (1996), 29(11), 2750-2753.

### Organic Layer Deposition

Organic layers as described herein including, without limitation, hole-injection layers, hole-transporting layers, first light-emitting layers, electron-transporting layers, electron injection layers and layers comprising a compound of formula (I) may each independently be deposited by evaporation or by a solution deposition method from a solution in one or more solvents.

Suitable methods for solution deposition as disclosed anywhere herein are, without limitation, printing and coating techniques such spin-coating, inkjet printing, screen printing, gravure printing and flexographic printing.

Preferably, the first light-emitting layer is deposited from a solution.

Preferably, hole-injection layers, hole-transporting layers, electron-transporting layers and electron injection layers, where present, are deposited from a solution.

Preferably, the compound of formula (I) is deposited by evaporation.

The one or more solvents of a solution may be selected according to the solubility of the material or materials to be deposited. Exemplary non-polar solvent materials include benzenes substituted with one or more substituents selected from C₁₋₁₀ alkyl and C₁₋₁₀ alkoxy groups, for example toluene, xylenes and methylanisoles, and mixtures thereof. Exemplary polar solvents include, without limitation, protic solvents, optionally water or an alcohol; dimethylsulfoxide; propylene carbonate; or 2-butanone. Exemplary alcohols include methanol ethanol, propanol, butoxyethanol and monofluoro-, polyfluoro- or perfluoro-alcohols, optionally 2,2,3,3,4,4,5,5-octafluoro-1-pentanol.

In a solution deposition method, the solvent may be removed following deposition of the solution by heating, alone or with other methods such as vacuum treatment. In the case where a layer is formed over the compound of formula (I) by a solution deposition method, for example an electron-transporting layer or an electron injection layer, the heating of the partially formed device as described herein may also be used to evaporate the solvent of the solution deposited over the compound of formula (I).

### Examples

### Measurements

HOMO and LUMO levels as described anywhere herein are as measured by square wave voltammetry.

### Equipment:

CHI660D Electrochemical workstation with software (IJ Cambria Scientific Ltd))

### CHI 104 3 mm Glassy Carbon Disk Working Electrode (IJ Cambria Scientific Ltd))

Platinum wire auxiliary electrode

### Reference Electrode (Ag/AgCl) (Havard Apparatus Ltd)

### Chemicals

| | |
|---|---|
| Acetonitrile | (Cell solution solvent) |
| (Hi-dry anhydrous grade-ROMIL) | |
| Toluene | (Sample preparation solvent) |
| (Hi-dry anhydrous grade) | |
| Ferrocene - FLUKA | (Reference standard) |
| Tetrabutylammonium- | (Cell solution salt) |
| hexafluorophosphate- FLUKA) | |

### Sample Preparation

The acceptor polymers were spun as thin films (˜20 nm) onto the working electrode; the dopant material was measured as a dilute solution (0.3 w %) in toluene.

### Electrochemical Cell

The measurement cell contains the electrolyte, a glassy carbon working electrode onto which the sample is coated as a thin film, a platinum counter electrode, and a Ag/AgCl reference glass electrode. Ferrocene is added into the cell at the end of the experiment as reference material (LUMO (ferrocene)=−4.8 eV).

### Materials

Devices were made using materials set out in Table 1

TABLE 1

| Material name | Structure |
|---|---|
| Compound (I)-1 | 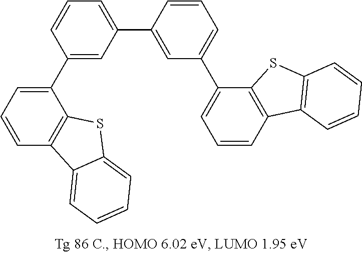 [SMILES: c1cc(-c2cccc(-c3cccc4c3sc3ccccc34)c2)cc(-c2cccc3c2sc2ccccc23)c1] |
| Host 1 | 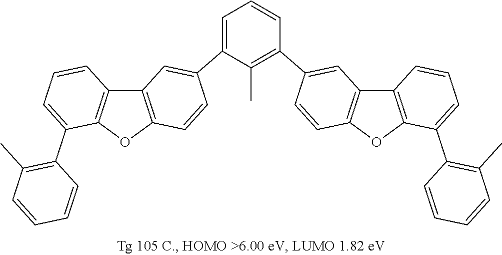 |
| Green Phosphorescent Emitter 1 | 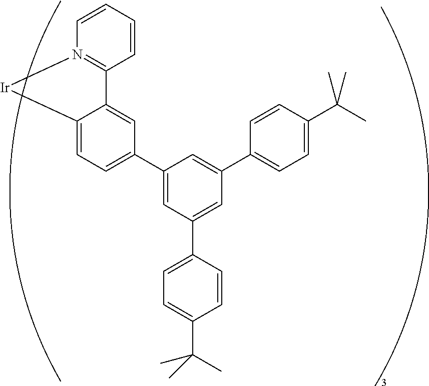 |
| Blue Phosphorescent Emitter 1 | 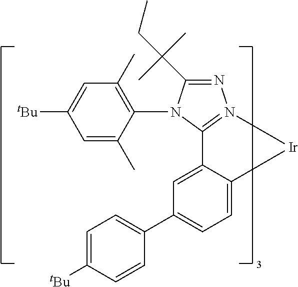 |
| Red Phosphorescent Repeat Unit 1 | 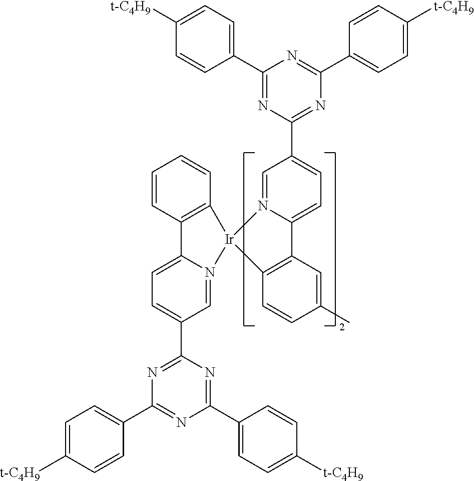 |
| Electron-Transporting Repeat Unit 1 | 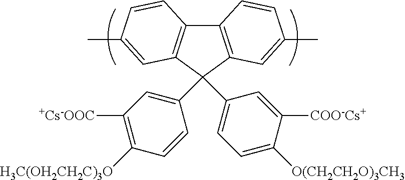 |

### Device Example 1

A substrate carrying ITO (45 nm) was cleaned using UV/Ozone. A hole injection layer was formed to a thickness of about 35 nm by spin-coating a formulation of a hole-injection material available from Nissan Chemical Industries. A red light-emitting layer was formed to a thickness of about 20 nm by spin-coating a red-emitting hole-transporting polymer comprising fluorene repeat units, amine repeat units and Red Phosphorescent Repeat Unit 1 and substituted with crosslinkable groups, and crosslinking the polymer by heating at 180° C. The green and blue light-emitting layer was formed to a thickness of about 70 nm by spin-coating Host 1 (74 wt %), Green Phosphorescent Emitter (1 wt %) and Blue Phosphorescent Emitter 1 (25 wt %). A layer of Compound (I)-1 was evaporated onto the light-emitting layer. An electron-transporting layer was formed by spin-coating a polymer comprising Electron-Transporting Unit 1 onto the layer of Compound (I)-1 from a 2,2,3,3,4,4,5,5-octafluoro-1-pentanol solution. This partially formed device was heated to 130-150° C. on a hotplate. A cathode was formed by evaporating a layer of sodium fluoride of about 2 nm thickness, a layer of aluminium of about 100 nm thickness and a layer of silver of about 100 nm thickness.

### Comparative Device 1

A device was prepared as described in Device Example 1 except that the layer comprising Compound (I)-1 was not formed.

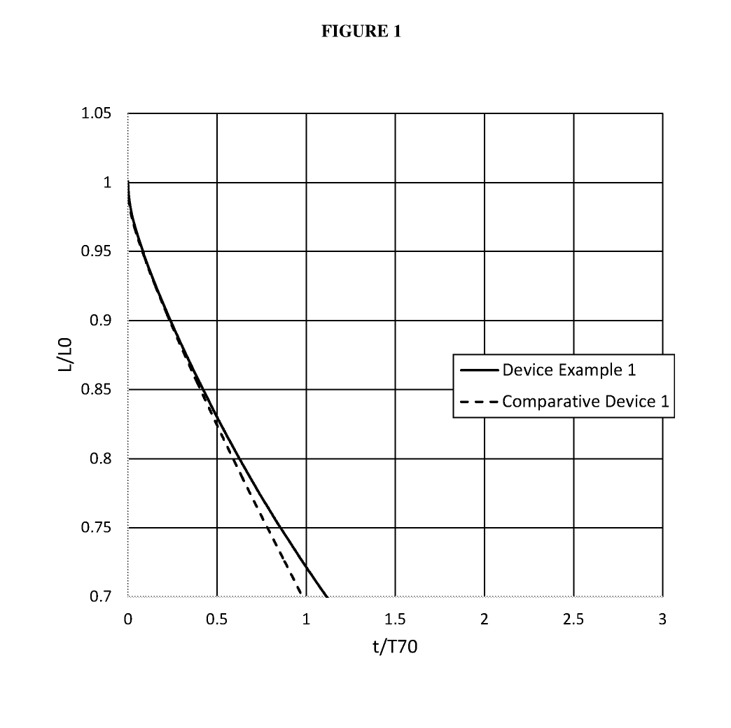

With reference to FIG. 1, lifetime of Device Example 1 is higher than that of Comparative Device 1.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A method of forming an organic light-emitting device comprising the steps of:
forming a first light-emitting layer over an anode;
forming a layer comprising a compound of formula (I) on the light-emitting layer to form a partially formed device:
(Ar)ₘ—(X)ₙ (I)
wherein Ar in each occurrence is independently a C₆₋₂₀ aromatic group; m is at least 1; n is at least 1; and X is a group of formula (II): 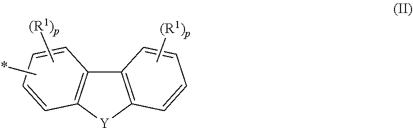
wherein Y is O or S; R¹ in each occurrence is independently a substituent; p is 0 or a positive integer; and * is a bond to Ar;
forming a cathode over the compound of formula (I); and
heating the partially formed device before and/or after formation of the cathode.

2. A method according to claim 1 wherein the partially formed device is heated to above the glass transition temperature (Tg) of a material of the first light-emitting layer.

3. A method according to claim 1 wherein the light-emitting layer comprises a host and a light-emitting dopant

4. A method according to claim 3 wherein the host comprises at least one unsubstituted or substituted dibenzothiazole or a dibenzofuran.

5. A method according to claim 3 wherein the difference in LUMO level of the host material and the compound of formula (I) is no more than about 0.2 eV.

6. A method according to claim 3 wherein the difference in HOMO level of the host material and the compound of formula (I) is no more than about 0.2 eV.

7. A method according to claim 3 wherein the partially formed device is heated to above the Tg of the host.

8. A method according to claim 1 wherein the partially formed device is heated to above the Tg of the compound of formula (I).

9. A method according to claim 1 wherein the partially formed device is heated to a temperature in the range of 70-150° C.

10. A method according to claim 1 wherein an electron-transporting layer or electron-injecting layer is deposited between the compound of formula (I) and the cathode.

11. A method according to claim 1 wherein the compound of formula (I) is selected from formulae (Ia)-(Id):

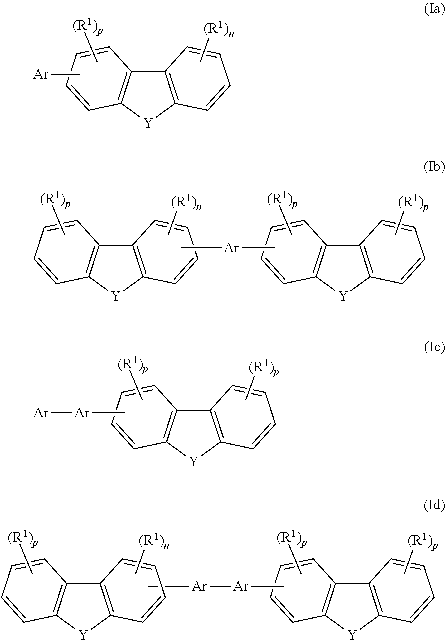

12. A method according to claim 1 wherein the group of formula (II) has formula (IIa):

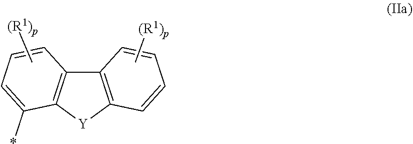

13. A method according to claim 1 wherein Ar is fluorene or phenyl, each of which may be unsubstituted or substituted with one or more substituents.

14. A method according to claim 13 wherein (Ar)ₘ is selected from formulae (IIIa)-(IIIf): 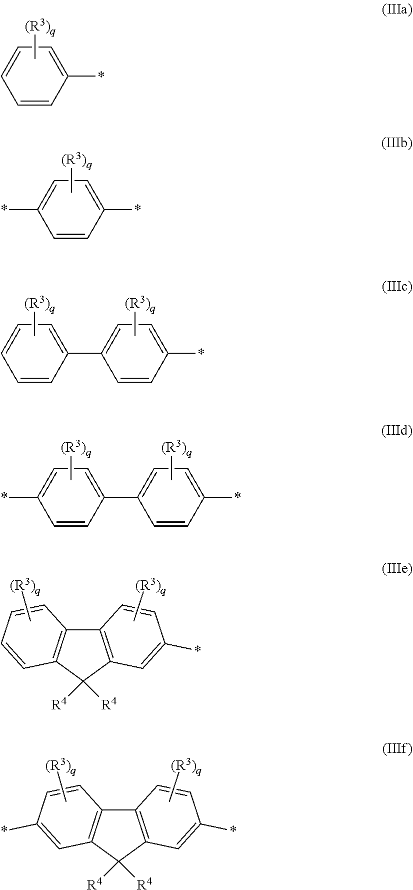
wherein R³ independently in each occurrence is a substituent; q is 0 or a positive integer; and R⁴ independently in each occurrence is a substituent wherein the two groups R⁴ may be linked to form an unsubstituted or substituted ring.

15. A method according to claim 1 wherein the first light-emitting layer is formed by depositing a solution comprising the or each component of the first light-emitting layer in a solvent.

16. A method according to claim 1 wherein the layer comprising a compound of formula (I) is formed by evaporation.

17. A method according to claim 1 wherein the device comprises at least one further light-emitting layer.

18. A method according to claim 1 wherein the device emits white light when in use.

19. An organic light-emitting device obtainable by a method according to claim 1.
